(19) United States
(12) Patent Application Publication
(10) Pub. No.: US 2007/0282098 A1
(43) Pub. Date: Dec. 6, 2007

(54) Process for combining parallel oligonucleotide synthesis

(76) Inventors: Jan Weiler, (US); Jorg Hohelsel, (US)

Correspondence Address: MOORE & VAN ALLEN PLLC, P.O. BOX 13706, Research Triangle Park, NC, 27709, US

(21) Appl. No.: 11/700,617

(22) Filed: Jan. 31, 2007

Related U.S. Application Data
(63) Continuation of application No. 09/202,969, filed on Nov. 3, 2003, now abandoned.

(30) Foreign Application Priority Data
Jun. 24, 2006 (DE) DE 19625397

(51) Int. Cl. C07H 21/00; C12Q 1/68

(52) U.S. Cl. 536/25.3; 422/50

(57) ABSTRACT

The invention concerns a process for the parallel synthesis of oligonucleotides on an alkylamino-modified matrix surface, characterized in that 3-succinate derivatives of protected nucleosides are applied thereto and oligonucleotide synthesis takes place by means of automated DNA synthesis. The invention also concerns the use of the oligomer chip formed in the process.

### CROSS-REFERENCE TO RELATED APPLICATIONS

This application is a Continuation Application of co-pending U.S. application Ser. No. 09/202,969 which claims the priority of International Patent Application No. PCT/DE97/01332 filed on Jun. 24, 1997, which in turn claims priority of German Patent Application No. DE 19625397 filed on Jun. 24, 1996.

### BACKGROUND OF THE INVENTION

1. Field of the Invention

This invention relates to a process for combining parallel oligonucleotide synthesis and preparation of oligomer chips.

2. Related Art

Stimulated by a combination of solid-phase technology and phosphoramidite chemistry, there was major progress in the automation of DNA synthesis. Today, the production of synthetic oligonucleotides used for biological, biomedical and physical applications takes place in automated DNA synthesis apparatuses. These apparatuses produce oligonucleotides within the nanomole to micromole range. However, two opposed trends regarding the oligomer synthesis have existed in the past few years. Clinical applications, such as the antisense strategy, require oligonucleotide amounts of grams or even kilograms, which makes necessary to raise the standard for the oligomer synthesis. In contrast thereto, only oligomer amounts within the picomole range are needed for applications in molecular biology, e.g. for the polymerase chain reaction or for DNA sequencing. However, a number of different oligonucleotides are required for these applications. This raised the problem of having to produce different oligomers in small amounts at the same time. On the other hand, what is called the “oligomer chip technology” plays a more and more important part for the diagnostic DNA analysis in molecular biology. Also with respect to many methods of genomic analysis, e.g. for “sequencing by hybridization” the use of matrices onto which regular oligonucleotide frames were applied, involves a great potential.

### SUMMARY OF THE INVENTION

Therefore, it is the object of the present invention to provide a process by which it is possible to provide oligonucleotides as different as possible in simple manner, which, on the one hand, may serve as “oligomer chips” for hybridization experiments and, on the other hand, can be removed regularly and are thus available for molecular biological reactions such a PCR or DNA sequencing.

This object is achieved by a process according to claim 1. Preferred embodiments follow from the subclaims.

The inventors based the development of their new process on the “oligomer chip technology” (Southern, E. M. et al., Genomics 13, 1008-1017; Caviani-Pease, A. C. et al., Proc. Natl. Acad. Sci., U.S.A. 91, 5022-5026) which has been used for DNA sequencing so far. This technique uses a matrix having short oligonucleotide sequences bonded thereto as an objective of hybridization experiments. In this connection, attention is paid to the fact that the oligonucleotides bonded to the matrix are firmly bonded thereto.

The inventors have now modified this bond of the oligonucleotides to the matrix such that, on the one hand, it is possible to use the resulting oligomer chip for hybridization analyses of DNA by means of oligomer chip technology or to remove regularly the oligonucleotides so as to then use them for PCR purposes or in the enzymatic DNA sequencing and as a probe for hybridizations, respectively.

A polymer sheet or a glass surface can be used as a matrix on which the oligonucleotides are bonded. However, an aminated polypropylene sheet which is further surface-modified, is preferred. A possible surface modification is the attachment of alkylamino groups, preferably methylamino groups. For example, this is effected such that an aminated polypropylene sheet is shaken in a mixture of dry dichloromethane, dry dioxan, p-nitrophenylchloroformate and triethylamine for several hours. After washing with dichioromethane, the sheet is shaken in a mixture of pyridine and acetic anhydride for several hours, so that amino groups left on the surface are saturated. Thereafter, the sheet is washed with dichloromethane and taken up in acetonitrile or dimethylformamide. 1, 6-Bis (methylamino)-hexane is added, and the mixture is shaken f or several hours to several days. After washing using DMF, methanol and acetone, the methylamino-modified membrane is dried.

An above matrix, particularly a methylamino-modified membrane, can be fixed in a synthesis chamber, e.g. one as shown in FIG. 1. 3′-Succinate derivatives of protected nucleosides (dA^{NPEOC}, dC^{NPEOC}, dG^{NPEOC/NPE}, dT and fluorescein-labeled dC) are applied onto the matrix, the 3′-succinate derivatives being producible as described in Kierzek et al. (Biochemistry 25, pp. 7840-7846 (1986)). For the application, desired nucleoside-3′-succinates are mixed with an organic solvent, e.g. N-methylmorpholine and acetonitrile, before O-[(ethoxycarbonyl) cyanomethyleneamino]-N,N,N¹,N¹,-tetramethyluroniumtetrafluoroborate (TOTU) is added. This reaction mixture is fed into the synthesis chamber and a simple given cycle is activated in the chamber. This cycle is e.g. such that the rows on the matrix, preferably the methylamino-modified polypropylene sheet, are moistened with the reaction mixture and incubated f or a certain time, e.g. 30 minutes. Then, the reagents are rinsed and the rows are washed several times with an organic solvent, such as acetonitrile. For blocking the rest of the methylamino groups on the matrix, the latter is treated with a mixture of pyridine and acetic anhydride for several hours. As an alternative, the matrix removed from the synthesis chamber can be treated in a mixture of acetic anhydride, pyridine and N-methylimidazole. After being washed with DMF, methanol and acetone, the matrix is dried.

It can be favorable for the oligonucleotide synthesis to use the below scheme. For example, the synthesis chamber shown in FIG. 1 is suited for carrying out this synthesis. Modifications of the scheme as regards the sequence of steps, exchange of the reagents and solvents, respectively, as well as incubation periods are within the scope of the person skilled in the art, and the scheme shall not at all be interpreted or regarded as limiting the invention.

TABLE
Scheme of an oligonucleotide synthesis

| Step | Reagent or solvent | Time (s) |
|---|---|---|
| Washing | acetonitrile | 30 |
| Detritylation | 3% TCA in dichloromethane | 80 |
| Washing | acetonitrile | 210 |
| Coupling | 75 mM phosphoramidite + 0.5 M | 40 |
| | tetrazole in acetonitrile | |
| Washing | acetonitrile | 30 |
| Removal | acetic anhydride/N- | 50 |
| | methylimidazole in acetonitrile | |
| Oxidation | 1 M t-butylhydroperoxide in | 130 |
| | acetonitrile | |
| Washing | acetonitrile | 120 |

When the synthesis is complete, the matrix is removed from the synthesis chamber and shaken in 1 M 1, 8-diazabicyclo-(5.4.0)-undec-7-ene (DBU) in acetonitrile for deprotection of the oligonucleotides. Before it is used in hybridization experiments or the oligomers are removed, the matrix is washed, e.g. with acetonitrile and acetone.

For removing the individual oligonucleotides, the desired surface area of the resulting oligomer chip is excised and, following incubation in 30% aqueous ammonia for several hours, e.g. 2 hours, the removed oligonucleotide products are lyophilized and used for PCR or DNA sequencing methods.

In the above described NPE/NPEOC strategy, the β-eliminating base protecting groups 2-nitrophenylethyl (NPE) and 2-(4-nitrophenyl) ethoxycarbonyl (NPEOC) are used. These functions permit the deprotection of the biopolymer by the strong but non-nucleophilic DBU base, while the oligonucleotides remain attached to the matrix.

The present invention is now described in more detail with reference to the following illustrations.

### BRIEF DESCRIPTION OF THE FIGURES

FIG. 1: Reaction chamber for the oligonucleotide synthesis on a polypropylene sheet, the apparatus being adjusted such that it permits 90° rotation of the polypropylene sheet between the synthesis steps.

FIG. 2: Attachment of a sample and removal by ammonia (NH₄OH), used for the standard synthesis of free oligonucleotides (a) and for the production of oligonucleotide arrays (b); X=O; X=NH.

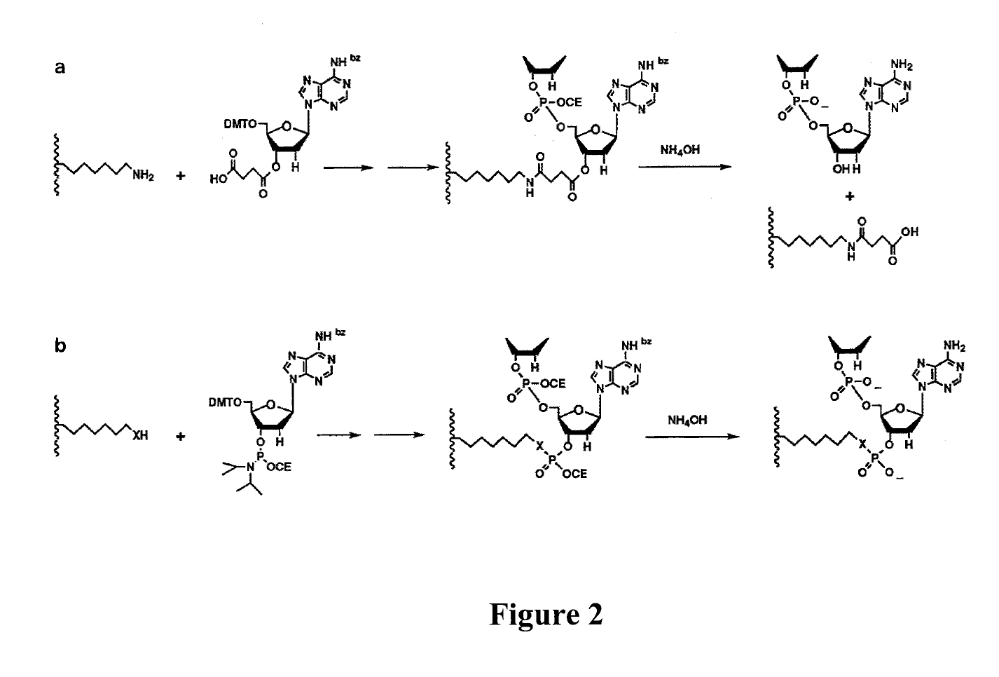

FIG. 3: Removal of the solid phase-bonded oligonucleotides from the surface. Application of the “NPE/NPEOC strategy” permits the alternative use of the oligomer chips for either hybridization experiments (option I) or as a source for the isolation of individual primer molecules (option II).

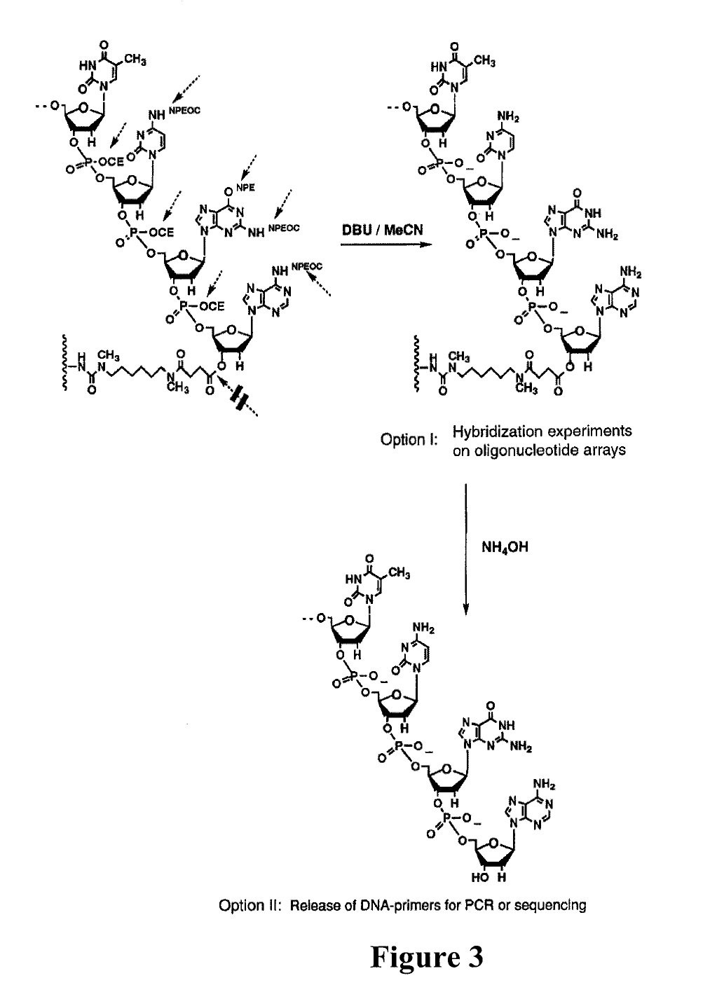

FIG. 4: Activation of the methylamino-modified polypropylene sheet and linkage and coupling of the suitably protected nucleosides.

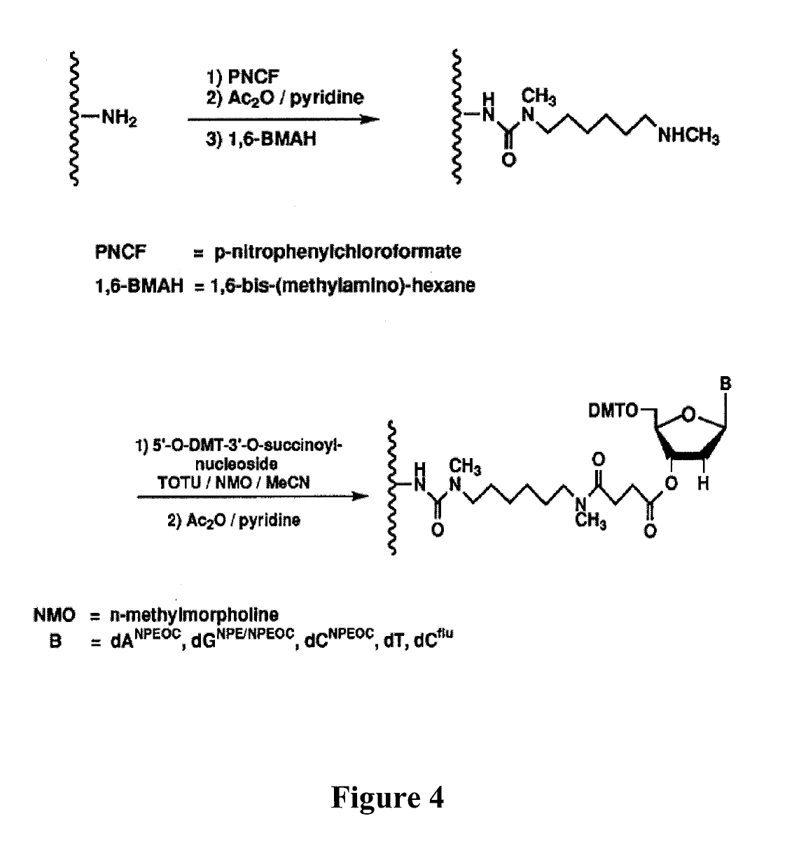

FIG. 5: Exemplary hybridizations with oligomer arrays on polypropylene membranes. (a), (b): 4 different starting nucleosides, dC^{NPEOC} (columns 3, 7), dA^{NPEOC} (columns 2, 6), dC^{bz} (column 1) and dC^{fl} (columns, 4, 8) were applied to a polypropylene sheet using an 8-channel synthesis apparatus. The sheet was then removed from the reaction chamber and turned by 90°. 7 different oligonucleotides were then synthesized (A-H), which produce 49 sites having 28 different sequences. A channel was left empty in both dimensions as control (lanes 5 and D). After the oligomer deprotection some sites (e.g. B2, 6, 8; C8; E8) were excised for the purpose of analysis. The remaining sheet was hybridized with a (a) 9-mer d(CTATAGTGA) and (b) a 12-mer d(GA₁₁). Complementary sequences are underlined. In (c) and (d) the sheet was turned by 90° between the synthesis steps. Nonamers were hybridized whose sequences cover breaking points of the synthesis.

FIG. 6: PCR amplification of a plasmid insert. Both commercial primers were used for the reaction, plotted in lane 1. In lanes B and C each primer was replaced by oligonucleotides which were isolated from a 0.16 cm² piece of the polypropylene sheet treated according to the invention. Marker (lane D) is HindIII-digested λ-DNA and plasmid pUC18 DNA excised with FspI.

### DETAILED DESCRIPTION OF THE INVENTION

### EXAMPLE

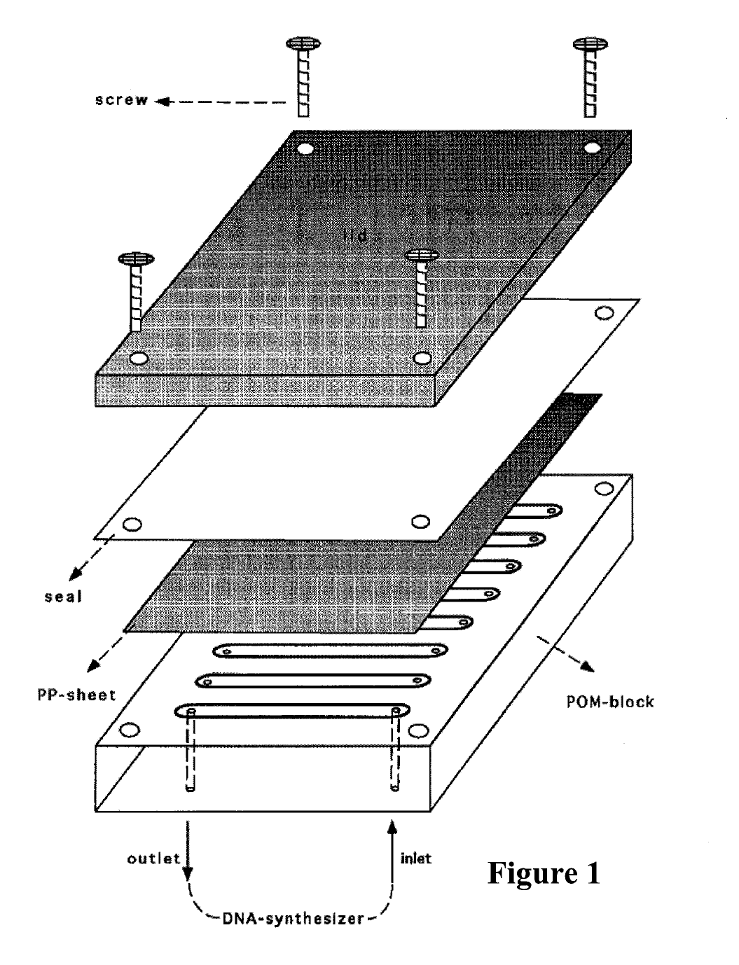

A polyoxymethylene (POM) block was prepared such that it contains 8 channels, each having a depth of 1 mm, a length of 70 mm, and a width of 4 mm and 2 holes at both ends for connection to the inlet and outlet of a conventional DNA synthesis apparatus. The polypropylene film was kept in place by a silicone seal and a perspex cover screwed on the POM base, as shown in FIG. 1. In order to obtain a perfect seal, an additional pressure was applied to the entire apparatus by means of a clamp. Owing to the removal of the polypropylene sheet after one or several cycles, rotation by 90° and continued synthesis, this simple and small instrument permits the synthesis of up to 64 different oligomers. An aminated polypropylene sheet (8×8 cm²) was slightly shaken in a mixture of 25 ml dry dichioromethane, 25 ml dry dioxan, 0.2 g p-nitrophenyl chloroformate and 160 μl triethylamine at room temperature for 2 h. After being washed with dichloromethane, the sheet was shaken in a 1:1 mixture of pyridine and acetic anhydride for 2 h thereby saturating the amino groups left on the surface. Thereafter, the sheet was washed with dichloromethane and taken up in 50 ml acetonitrile or dimethylformamide (DMF) 0.2 ml 1,6-bis-(methylamino) hexane were added, and the mixture was shaken at 40° C. for 48 h. After successive washing with DMF, methanol and acetone, the methylamino-modified propylene sheet was dried and stored at 4° C.

3′-succinate derivatives of protected nucleosides (dA^{NPEOC}, dC^{NPEOC}, dG^{NPEOC/NPE}, dT and fluorescein-labeled dC (=dC^{fl}) were produced by the method described by Kierzek et al. (Biochemistry 25, pp. 7840-7846 (1986)) and applied onto a methylamino-modified polypropylene sheet after fixing the sheet in a polyoxymethylene block. For this purpose, 5 mg of the desired nucleoside-3′ succinate were mixed with 25 μl N-methylmorpholine and 10 ml acetonitrile before 4 mg TOTU were added. The bottle with the mixture was immediately connected to a DNA synthesis apparatus and a. simple pre-programmed cycle was activated in the apparatus: First, the rows were wet with the reaction mixture, and the reaction was incubated for 30 min. Then, the reagents were washed away using argon, and the rows were washed several times with acetonitrile. In order to block the rest of the methylamino groups on the polypropylene sheet, acetic anhydride and N-methylimidazol in acetonitrile were applied. As an alternative, the derivatized polypropylene membranes were removed from the chamber and shaken in a mixture of 10 ml acetic anhydride, 10 ml dry pyridine and 1 ml N-methylimidazole in a polypropylene box for 2 h. After subsequent washing with DMF, methanol and acetone, the sheets were dried and stored at 4° C. until they were used.

The oligonucleotide synthesis was carried out on the polypropylene sheet as follows from the above Table 1. After the synthesis was complete, the sheets were removed from the chamber and shaken in 1 M-DBU in acetonitrile in a polypropylene box at 40° C. overnight. The membrane was washed with acetonitrile and acetone before it was used for either hybridization experiments or the removal of the oligomers.

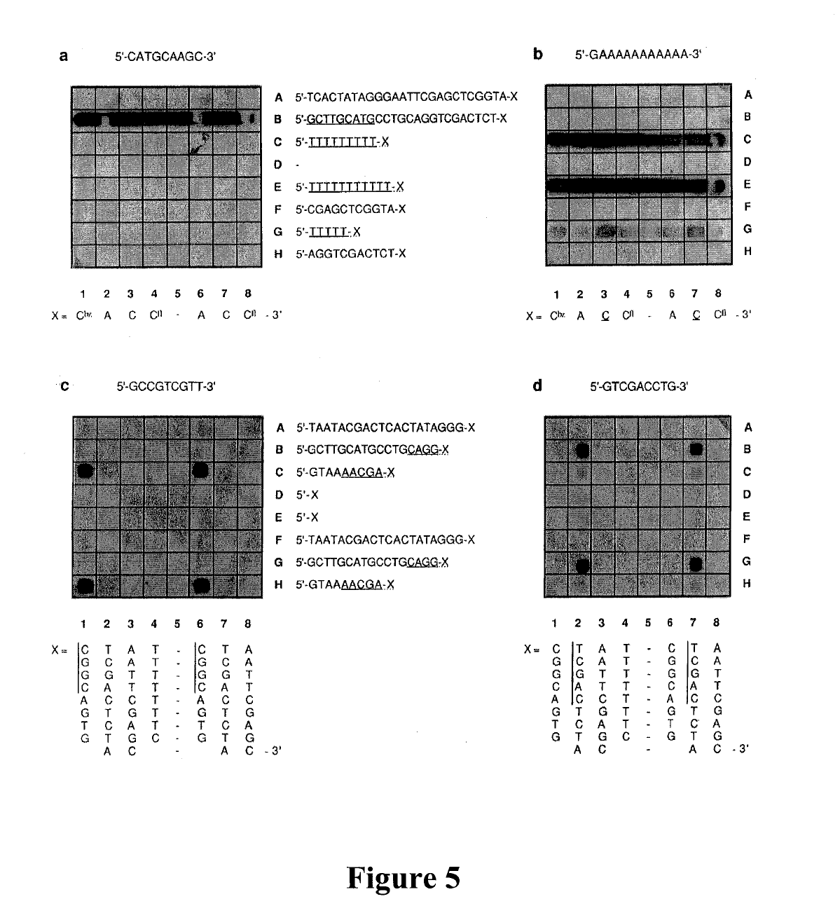

Oligonucleotide probes were end-labeled with [γ³²P]ATP under standard conditions. The oligomer arrays were pre-hybridized in 600 mM NaCl, 60 mM sodium citrate, pH 7.5, 7.2% sodium-N-lauroylsarcosine for 1 h and then incubated in 10 ml of the same solution which included about 1 Mcpm radioactively labeled oligomer probe (concentration=6 picomoles/ml) at 4° C., for 18 h. After 30 minutes of washing at 4° C., autoradiophraphy was carried out at -70° C. The probes were removed from the sheets by incubation in hybridization buffers at 65° C. for 3 h. The results of the hybridzations are shown in FIG. 5 particularly FIGS. 5A and 5B.

For removing the single oligonucleotides, the desired sites of the oligomer array on the polypropylene sheet were excised. After incubation in 30% acqueous ammonia for 2 h, the removed oligonucleotide products were lyophilized and used for PCR and DNA sequencing experiments without further purification.

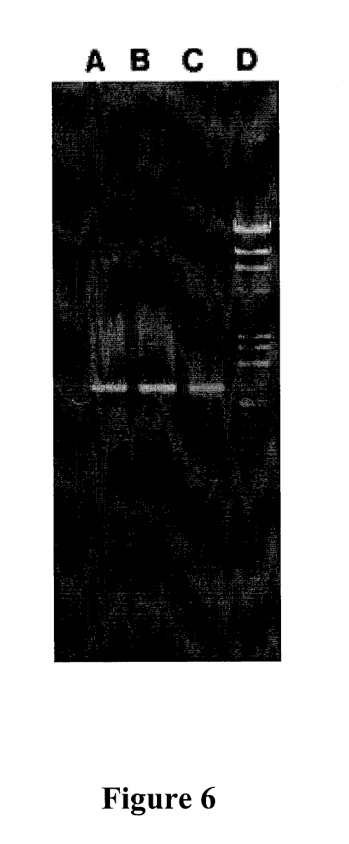

In order to test the suitability of the oligomers for PCR, a PCR was carried out with the recombinant plasmid pTZ18R under standard conditions, as described earlier (Scholler et al., Nucleic Acids Res. 23, pp. 3842-3849, (1995)). Primers were 26-mers and 29-mers which bind to the vector directly adjacent to each side of the insert DNA. An oligonucleotide amount which corresponded to a polypropylene surface area of 0.16 cm², was used in the reactions with 25 μl volume. PCR was carried out: Primer annealing and extension at 68° C., strand denaturation at 95° C. On an agarose gel, the products were compared with the results obtained with common commercial primer molecules used in a concentration of 1 μM. As follows from FIG. 6, no significant difference with respect to quality and quantity of the PCR products resulted.

The use in an enzymatic sequencing reaction also showed that the oligomers obtained and removed according to the invention show no significant quality differences over purchasable ones.

1. A process for the parallel synthesis of oligonucleotides on an alkylamino-modified matrix surface, characterized in that 3-succinate derivatives of protected nucleosides are attached thereto and oligonucleotide synthesis takes place by means of automated DNA synthesis.

2. The process according to claim 1, where the matrix surface is modified with methylamino groups.

3. The process according to claim 1, wherein the 3′ succinate derivatives are from dA^{NPEOC}, dC^{NPEOC}, dG^{NPEOC/NPE}, dT and/or fluorescein-labeled cD.

4. The process according to claim 2, wherein the 3′ succinate derivatives are from dA^{NPEOC}, dC^{NPEOC}, dG^{NPEOC/NPE}, dT and/or fluorescein-labeled cD.

5. The process according to claim 1, where the matrix is fixed in a synthesis chamber having several channels.

6. The process according to claim 2, where the matrix is fixed in a synthesis chamber having several channels.

7. The process according to claim 3, where the matrix is fixed in a synthesis chamber having several channels.

8. The process according to claim 1, wherein the matrix is made of glass or a polymer.

9. The process according to claim 8, wherein the polymer is polypropylene.

10. An oligomer chip comprising:
an alkylamino-modified matrix; and
3′ succinate derivatives selected from the group consisting of dA^{NPEOC}, dC^{NPEOC}, dG^{NPEOC/NPE}, dT and fluorescein-labeled cD applied to the alkylamino-modified matrix.

11. The oligomer chip according to claim 10, for use in a DNA synthesis chamber.


SEQUENCE LISTING (2 sequences)

SEQ ID NO: 1 - DNA, 9 bp, Artificial Sequence
  Synthetic Construct
ctatagtga

SEQ ID NO: 2 - DNA, 12 bp, Artificial Sequence
  Synthetic Construct
gaaaaaaaaaaa